(19) United States
(12) Patent Application Publication
(10) Pub. No.: US 2014/0199292 A1
(43) Pub. Date: Jul. 17, 2014

(54) COMPOSITIONS AND METHODS FOR INHIBITING TUMOR DEVELOPMENT CAUSED BY CHEMOTHERAPY INDUCED SENESCENCE

(71) Applicants: Corine Bertolotto-Ballotti, Nice cedex 3 (FR); Robert Ballotti, Nice cedex 3 (FR); Mickael Ohanna, Nice cedex 3 (FR); Sandy Giuliano, Nice cedex 3 (FR)

(72) Inventors: Corine Bertolotto-Ballotti, Nice cedex 3 (FR); Robert Ballotti, Nice cedex 3 (FR); Mickael Ohanna, Nice cedex 3 (FR); Sandy Giuliano, Nice cedex 3 (FR)

(73) Assignee: INSERM (Institut National de la Sante et de la Recherche Medicale), Paris (FR)

(21) Appl. No.: 14/000,663

(22) PCT Filed: Mar. 14, 2012
(86) PCT No.: PCT/EP2012/054482

§ 371 Date: Dec. 16, 2013

(30) Foreign Application Priority Data
Mar. 15, 2011 (EP) 11305284.9

(51) Int. Cl. A61K 31/713; G01N 33/574; C12Q 1/68; A61K 39/395; A61K 45/06

(52) U.S. Cl. CPC A61K 31/713; A61K 39/39558; A61K 45/06; C12Q 1/6886; G01N 33/574
USPC 424/133.1; 514/44 A; 435/6.12; 435/7.1; 435/7.92

(57) ABSTRACT

The present invention relates to products and compositions containing (i) a chemotherapeutic agent and (ii) at least an active compound chosen from CCL2 inhibitors, CCR2 inhibitors, NF-κB inhibitors, PARP-1 inhibitors and ATM inhibitors, as a combined preparation for simultaneous, separate or sequential use for inhibiting tumor development caused by tumor cell senescence induced by said chemotherapeutic agent. The invention also refers to a method for monitoring the response to a chemotherapeutic agent of a patient suffering from a cancer, and to a method for predicting the tumor size evolution and/or the onset of metastasis in a patient suffering from a cancer.

The present application is filed pursuant to 35 U.S.C. 371 as a U.S. National Phase application of International Patent Application No. PCT/EP2012/054482, which was filed Mar. 14, 2012, claiming the benefit of priority to European Patent Application No. 11305284.9, which was filed on Mar. 15, 2011. The entire text of the aforementioned applications is incorporated herein by reference in its entirety.

### FIELD OF THE INVENTION

The present invention relates to products and compositions for inhibiting tumor development caused by chemotherapy induced senescence. The invention also refers to a method for monitoring the response to a chemotherapeutic agent of a patient suffering from a cancer, and to a method for predicting the tumor size evolution and/or the onset of metastasis in a patient suffering from a cancer, said cancer being preferably a melanoma.

### BACKGROUND OF THE INVENTION

Melanoma is a malignant tumor of melanocytes. It occurs less commonly than other skin cancers, but its evolution is much more dangerous. The treatment of melanoma includes surgical removal of the tumor, chemo- and immunotherapy, or radiation therapy. It is better treated as it is early diagnosed.

Melanoma cells are well-known for their resistance to apoptotic stimuli, likely a remnant of the melanocyte status from which they derive. Indeed, melanocytes have evolved potent anti-apoptotic mechanisms required to resist to the mortal effect of ultraviolet radiation constantly reaching the skin allowing them to achieve their physiologic protective function by synthesizing melanin. Apoptotic resistance represents an important cause that limits the efficacy of the anti-melanoma therapies developed so far. In addition to apoptosis, cellular senescence is another important cellular failsafe mechanism. Ordinarily, senescence is a process arising in normal cells in response to telomere erosion or to oncogenic stress acting, through checkpoint activation and cell cycle arrest, as a barrier to tumorigenesis. Activation of checkpoint proteins prevents the replication of genomically instable cells, considered as the precursors of transformed cells (Hartwell and Kastan 1994; Elledge 1996). Surprisingly, several lines of evidence recently indicated that cellular senescence remains latently functional and can be reactivated in cancer cells, including melanoma cells (Giuliano et al. 2010b), lending strong support to the use of senescence induction as a therapeutic strategy against melanoma.

Senescent cells are growth arrested but remains metabolically active and can develop a secretory profile mainly composed of growth factors, cytokines and proteinases, a typical signature termed the senescence-associated secretory phenotype (SASP) or the senescence messaging secretome (SMS) (Coppe et al. 2008; Kuilman and Peeper 2009). Some of these factors can display cell autonomous activity and work to reinforce the senescent program (Acosta et al. 2008; Kuilman et al. 2008). Other secreted molecules exhibit cell-nonautonomous functions associated with inflammation and malignancy and act as pro-tumoral factors (Krtolica et al. 2001; Bavik et al. 2006; Liu and Hornsby 2007). These observations indicate that cellular senescence does not only function as a potent tumor suppressive process but may also exhibit deleterious effects.

As some chemotherapeutic drugs function in part through senescence induction (te Poele et al. 2002; Mhaidat et al. 2007), it would be useful to identify molecules which would be able to prevent its deleterious effects. Therefore, identifying said molecules would be of great interest. Particularly, said molecules would help in delaying or preventing the adverse effects of cellular senescence in chemotherapy treated patients suffering from cancers. They would allow a much better efficient treatment of cancers.

Surprisingly, the inventors showed that senescent melanoma cells express a senescence-associated secretory phenotype that can alter the behavior of nearby cells. Said secretome displays the following features:
it has pro-invasive properties;
it comprises different pro-inflammatory factors, among which the chemokine CCL2 is showed as a critical component; and
it is a poly(ADP-ribose) polymerase-1 (PARP-1) and nuclear factor-kappaB (NF-kB)-associated secretome (PNAS), observed in melanoma and also in non-melanoma cells.

Thus, inhibiting said PNAS and all its components is a good target for preventing or delaying the adverse effects of cellular senescence in chemotherapy treated patients suffering from cancers.

### SUMMARY OF THE INVENTION

The invention relates to a method for inhibiting tumor development caused by tumor cell senescence induced by a chemotherapeutic agent, in a patient suffering from a cancer and being treated by said chemotherapeutic agent, comprising the administration of (i) a chemotherapeutic agent and (ii) at least an active compound chosen from CCL2 inhibitors, CCR2 inhibitors, NF-κB inhibitors, PARP-1 inhibitors and ATM inhibitors, to said patient. The invention also relates to a product containing (i) a chemotherapeutic agent and (ii) at least an active compound chosen from CCL2 inhibitors, CCR2 inhibitors, NF-κB inhibitors, PARP-1 inhibitors and ATM inhibitors, as a combined preparation for simultaneous, separate or sequential use for inhibiting tumor development caused by tumor cell senescence induced by said chemotherapeutic agent.

Also provided is a pharmaceutical composition comprising (i) a chemotherapeutic agent and (ii) at least an active compound chosen from CCL2 inhibitors, CCR2 inhibitors, NF-κB inhibitors, PARP-1 inhibitors and ATM inhibitors, for use for inhibiting tumor development caused by tumor cell senescence induced by said chemotherapeutic agent.

The present invention also relates to a method for monitoring the response to a chemotherapeutic agent of a patient suffering from a cancer, comprising the step of measuring the level of expression of at least one gene selected from the group consisting of CCL2, Cyr61, IL6, IGFBP7, CCL8, PAI1, OPG, MMP2 and UPAR genes in cancer cells of said patient. Preferably, said method comprises:
a. treating said patient with a chemotherapeutic agent for a time period of at least 3 weeks; then
b. measuring the level of expression of at least one gene selected from the group consisting of CCL2, Cyr61, IL6, IGFBP7, CCL8, PAI1, OPG, MMP2 and UPAR genes in cancer cells of said patient, and
c. comparing the level of expression obtained in b. to a threshold value.

The present invention also relates to a method for predicting the tumor size evolution and/or the onset of metastasis in a patient suffering from a cancer, comprising the step of measuring the level of expression of at least one gene selected from the group consisting of CCL2, Cyr61, IL6, IGFBP7, CCL8, PAI1, OPG, MMP2 and UPAR genes in cancer cells of said patient.

Preferably according to the invention, said patient is suffering from a melanoma.

### DETAILED DESCRIPTION OF THE INVENTION

### Definitions

Chemokine ligand 2 (CCL2) is a small cytokine belonging to the CC chemokine family that is also known as monocyte chemotactic protein-1 (MCP-1) and small inducible cytokine A2. CCL2 recruits monocytes, memory T cells, and dendritic cells to sites of tissue injury, infection, and inflammation. It binds to its receptor CCR2.

As used herein, the term “CCL2 inhibitor” refers to compounds which inhibit signalling through CCL2, as well as compounds which inhibit the expression of the CCL2 gene. Compounds which inhibit signalling through CCL2 are called CCL2 antagonists. They include compounds which inhibit the activity of CCL2, by directly binding to CCL2, or by inhibiting CCL2 signalling by other mechanisms. CCL2 antagonists include, but are not limited to, small organic molecules, antibodies and aptamers.

Typically, CCL2 antagonists are murine antibodies, monoclonal or polyclonal.

As used herein, the term “CCR2 inhibitor” refers to compounds which inhibit signalling through CCR2, as well as compounds which inhibit the expression of the CCR2 gene.

Compounds which inhibit signalling through CCR2 are called CCR2 antagonists. They include compounds which inhibit the activity of CCR2, for example by directly binding to CCR2 and by inhibiting the CCL2 signalling, or by inhibiting CCR2 signalling by other mechanisms. CCR2 antagonists include, but are not limited to, small organic molecules, antibodies and aptamers.

As used herein, the term “NF-κB inhibitor” refers to compounds which inhibit signalling through NF-κB, as well as compounds which inhibit the expression of the NF-κB gene.

Compounds which inhibit signalling through NF-κB are called NF-κB antagonists. They include compounds which inhibit the activity of NF-κB, or by inhibiting NF-κB signalling by other mechanisms. NF-κB antagonists include, but are not limited to, small organic molecules, antibodies and aptamers.

Typically, NF-κB antagonists are murine antibodies, monoclonal or polyclonal, but also synthetic compounds such as sulfasalazine, BMS-345541 or bortezomid.

As used herein, the term “PARP-1 inhibitor” refers to compounds which inhibit signalling through PARP-1, as well as compounds which inhibit the expression of the PARP-1 gene. PARP-1 is an enzyme involved in DNA repair and programmed cell death.

Compounds which inhibit signalling through PARP-1 are called PARP-1 antagonists. They include compounds which inhibit the activity of PARP-1, or by inhibiting PARP-1 signalling by other mechanisms. PARP-1 antagonists include, but are not limited to, small organic molecules, antibodies and aptamers.

Typically, PARP-1 antagonists are synthetic compounds such as 3-aminobenzamide (3-AB), Iniparib (previously BSI 201), Olaparib (AZD-2281), ABT-888 (Veliparib), AG014699, CEP 9722, MK 4827, KU-0059436 (AZD2281), LT-673.

Ataxia telangiectasia mutated (ATM) is a serine/threonine kinase that plays a role in response to the DNA damage.

As used herein, the term “ATM inhibitor” refers to compounds which inhibit signalling through ATM, as well as compounds which inhibit the expression of the ATM gene. Compounds which inhibit signalling through ATM are called ATM antagonists. They include compounds which inhibit the activity of ATM, or by inhibiting ATM signalling by other mechanisms. ATM antagonists include, but are not limited to, small organic molecules, antibodies and aptamers.

Typically, ATM antagonists are KU55933 or caffeine.

Said inhibitors mentioned above may be specific. By “specific” or “selective” it is meant that the affinity of the antagonist for said target is at least 10-fold, preferably 25-fold, more preferably 100-fold, still preferably 500-fold higher than the affinity for other proteins.

CCL2, CCR2, NF-κB, PARP-1 and ATM inhibitors also include compounds which respectively inhibit the expression of the CCL2, CCR2, NF-κB, PARP-1 or ATM genes; said compounds are called inhibitors of gene expression. An “inhibitor of gene expression” refers to a natural or synthetic compound that has a biological effect to inhibit or significantly reduce the expression of said gene.

Consequently an inhibitor of CCL2 gene expression refers to a natural or synthetic compound that has a biological effect to inhibit or significantly reduce the expression of the CCL2 gene. An inhibitor of CCR2 gene expression refers to a natural or synthetic compound that has a biological effect to inhibit or significantly reduce the expression of the CCR2 gene. An inhibitor of NF-κB gene expression refers to a natural or synthetic compound that has a biological effect to inhibit or significantly reduce the expression of the NF-κB gene. An inhibitor of PARP-1 gene expression refers to a natural or synthetic compound that has a biological effect to inhibit or significantly reduce the expression of the PARP-1 gene. An inhibitor of ATM gene expression refers to a natural or synthetic compound that has a biological effect to inhibit or significantly reduce the expression of the ATM gene.

The inhibitors of gene expression include, but are not limited to, antisense oligonucleotides, siRNAs, shRNAs, ribozymes and DNAzymes.

Preferably, the inhibitors of gene expression are chosen from siRNAs.

As used herein, the term “cancer” refers to the pathological condition in mammals that is typically characterized by unregulated cell growth. Preferentially, the cancer is chosen from melanoma, breast cancer, prostate cancer, hepatocarcinome, bladder cancer, lung cancer, osteosarcoma and glioma. Preferably, the cancer is chosen from melanoma and breast cancer.

Preferably, the cancer is a melanoma.

As used herein, the term “patient” denotes a mammal, such as a rodent, a feline, a canine, and a primate. Preferably, a patient according to the invention is a human.

In the context of the invention, the term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies.

As used herein, the expression “anti-cancer agent” or “chemotherapeutic agent” refers to compounds which are used in the treatment of cancers.

Anti-cancer agents include but are not limited to temozolomide, fotemustine, dacarbazine, fludarabine, gemcitabine, capecitabine, methotrexate, taxol, taxotere, mercaptopurine, thioguanine, hydroxyurea, cytarabine, cyclophosphamide, ifosfamide, nitrosoureas, platinum complexes such as cisplatin, carboplatin and oxaliplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epimbicm, 5-fluorouracil, taxanes such as docetaxel and paclitaxel, leucovorin, levamisole, irinotecan (CPT-11), SN-38, estramustine, etoposide, nitrogen mustards, BCNU, nitrosoureas such as carmustme and lomustine, vinca alkaloids such as vinblastine, vincristine and vinorelbine, monoclonal antibodies against EGF receptor or VEGF, such as bevacizumab, cetuximab and panitumumab, imatimb mesylate, hexamethyhnelamine, topotecan, genistein, erbstatin, lavendustin and also bortezomib (also called PS341, and sold by Millenium Pharmaceuticals under the name Velcade).

Preferably, the anti-cancer agent is selected from temozolomide, fotemustine and dacarbazine.

As used herein, the term “senescence” refers to the phenomenon by which normal diploid cells loose the ability to divide under normal conditions. Senescence includes replicative senescence, oncogene-induced senescence and accelerated cellular senescence.

As used herein, the expression “tumor development caused by tumor cell senescence induced by a chemotherapeutic agent”, refers to the migration and proliferation of tumor cells in response to the PNAS (secretome) produced by senescent cells, said senescent cells being in senescence state because of the chemotherapeutic agent. Indeed, the treatment with a chemotherapeutic agent induces senescence of tumor cells; said senescent tumor cells then produce the PNAS, which in cascade stimulates the migration and proliferation properties of the remaining non injured tumor cells.

In a detailed embodiment, the treatment with a chemotherapeutic agent induces DNA damages in tumor cells, and PARP-1 and ATM activation, which upregulates NF-κB activity. This induces senescence in some of the tumor cells, which in response produce the PNAS, comprising CCL2. This PNAS stimulates the migration and proliferation properties of the remaining active (i.e. not senescent) tumor cells, thus inducing metastasis and tumor growth. This finally leads to a more aggressive cancer.

By “inhibiting the tumor development caused by tumor cell senescence induced by a chemotherapeutic agent”, it is meant preventing said tumor development in a patient.

### Therapeutic Compositions

The present invention relates to a product containing (i) a chemotherapeutic agent and (ii) at least an active compound chosen from CCL2 inhibitors, CCR2 inhibitors, CCR4 inhibitors, NF-κB inhibitors, PARP-1 inhibitors and ATM inhibitors, as a combined preparation for simultaneous, separate or sequential use for inhibiting tumor development caused by tumor cell senescence induced by said chemotherapeutic agent.

In this case, actives (i) and (ii) may be used in different compositions, and may be administered at different time intervals and via different ways.

The present invention also relates to a pharmaceutical composition comprising (i) a chemotherapeutic agent and (ii) at least an active compound chosen from CCL2 inhibitors, CCR2 inhibitors, NF-κB inhibitors, PARP-1 inhibitors and ATM inhibitors, for use for inhibiting tumor development caused by tumor cell senescence induced by said chemotherapeutic agent. In other terms, the present invention also relates to the association of (i) a chemotherapeutic agent and (ii) at least an active compound chosen from CCL2 inhibitors, CCR2 inhibitors, NF-κB inhibitors, PARP-1 inhibitors and ATM inhibitors, for preparing a medicament for inhibiting tumor development caused by tumor cell senescence induced by said chemotherapeutic agent.

Precisely, the NF-κB inhibitors, PARP-1 inhibitors and ATM inhibitors inhibit the onset of cell senescence, particularly the tumor cell senescence. On the other hand, the CCL2 inhibitors and CCR2 inhibitors inhibit the effects of the PNAS produced by the senescent tumor cells.

Preferably, said chemotherapeutic agent (i) is selected from temozolomide, fotemustine, dacarbazine, 5-fluorouracil, bevacizumab, irinotecan, SN38, oxaliplatin, cetuximab, panitumumab, leucovorine, bortezomib and capecitabine. More preferably, said chemotherapeutic agent (i) is selected from temozolomide, fotemustine and dacarbazine.

Preferably, said active compound (ii) is chosen from CCL2 antagonists, NF-κB antagonists, PARP-1 antagonists and ATM antagonists. More preferably, said active compound (ii) is chosen from CCL2 antagonists, CCL2 expression inhibitors, NF-κB expression inhibitors, PARP-1 expression inhibitors and ATM expression inhibitors. More preferably, said active compound (ii) is chosen from CCL2 siRNAs.

Preferably, the product used as a combined preparation and the composition according to the invention are used for inhibiting tumor growth and/or inhibiting tumor metastasis caused by tumor cell senescence, said senescence being induced by the chemotherapeutic agent administered to the patient.

Without wishing to be bound by theory, the inventors have discovered that said PNAS is secreted by senescent tumor cells in response to the chemotherapeutic agent, thus stimulating proliferation and migration of the remaining tumor cells. This phenomenon would lead to the increase of tumor growth and to metastasis, rendering the cancer more aggressive.

The present invention thus also relates to a product containing (i) a chemotherapeutic agent chosen from temozolomide, fotemustine and dacarbazine, and (ii) at least a CCL2 expression inhibitor, as a combined preparation for simultaneous, separate or sequential use for inhibiting melanoma development caused by melanoma cell senescence induced by said chemotherapeutic agent. The present invention also relates to a pharmaceutical composition comprising (i) a chemotherapeutic agent chosen from temozolomide, fotemustine and dacarbazine, and (ii) at least a CCL2 expression inhibitor, for use for inhibiting melanoma development caused by melanoma cell senescence induced by said chemotherapeutic agent.

Typically medicaments according to the invention comprise the active (ii), and optionally the active (i), together with a pharmaceutically-acceptable carrier. A person skilled in the art will be aware of suitable carriers. Suitable formulations for administration by any desired route may be prepared by standard methods, for example by reference to well-known text such as Remington; The Science and Practice of Pharmacy.

### Methods for Monitoring the Response to Treatment and for Predicting Evolution

The inventors surprisingly discovered that the expression of at least one of the genes of CCL2 and Cyr61, preferably both, was increased in senescent melanoma cells as compared to non senescent melanoma cells, in a patient treated with a chemotherapeutic agent. Thus, high levels of expression of said genes in some melanoma cells of a patient are indicative of their senescence state, and thus of their ability to secrete the PNAS and finally to stimulate the proliferation and migration properties of the non senescent melanoma cells. This would lead to melanoma growth and metastasis. In such a case, the treatment has to be re-evaluated, and the addition of a CCL2/NF-κB/PARP-1 or ATM inhibitor has to be considered.

The invention thus provides a method for monitoring the response to a chemotherapeutic agent of a patient suffering from a cancer, comprising the step of measuring the level of expression of at least one gene selected from the group consisting of CCL2, Cyr61, IL6, IGFBP7, CCL8, PAI1, OPG, MMP2 and UPAR genes in cancer cells of said patient. Preferably, the gene is selected from the group consisting of CCL2 and Cyr61.

Preferably, said method comprises:
a. treating said patient with a chemotherapeutic agent for a time period of at least 3 weeks; then
b. measuring the level of expression of at least one gene selected from the group consisting of CCL2, Cyr61, IL6, IGFBP7, CCL8, PAI1, OPG, MMP2 and UPAR genes in cancer cells of said patient, and
c. comparing the level of expression obtained in b. to a threshold value.

Preferably, said cancer is a melanoma.

The invention also provides a method for predicting the tumor size evolution and/or the onset of metastasis in a patient suffering from a cancer, comprising the step of measuring the level of expression of at least one gene selected from the group consisting of CCL2, Cyr61, IL6, IGFBP7, CCL8, PAI1, OPG, MMP2 and UPAR genes in cancer cells of said patient. Preferably, the gene is selected from the group consisting of CCL2 and Cyr61.

As used herein, the term “gene expression level” or “level of expression of a gene” refers to an amount or a concentration of a transcription product, for instance mRNA, or of a translation product, for instance a protein or polypeptide. Typically, a level of mRNA expression can be expressed in units such as transcripts per cell or nanograms per microgram of tissue. A level of a polypeptide can be expressed as nanograms per microgram of tissue or nanograms per milliliter of a culture medium, for example. Alternatively, relative units can be employed to describe a gene expression level.

As used herein, the expression of “measuring the level of expression of a gene” encompasses the step of measuring the quantity of a transcription product, preferably mRNA obtained through transcription of said gene, and/or the step of measuring the quantity of translation product, preferably the protein obtained through translation of said gene. Preferably, the step of measuring the expression of a gene refers to the step of measuring the quantity of mRNA obtained through transcription of said gene.

In one embodiment of the invention, the step of measuring the gene expression level is performed by the following method:

a) obtaining a biological sample comprising cancer cells from said patient,
b) measuring the level of expression of said gene(s) in said cancer cells in said biological sample.

According to the invention, in case of monitoring the response to a treatment of a patient suffering from melanoma, a biological sample may be a sample of the skin tissue or melanoma cells obtained from the patient according to methods known in the art. Said biological sample is for example a biopsy.

Typically, step b. of measuring the gene expression level may be performed according to the routine techniques, well known of the person skilled in the art.

More preferably, the measurement comprises contacting the cancer cells of the biological sample with selective reagents such as probes, primers, ligands or antibodies, and thereby detecting the presence of nucleic acids or proteins of interest originally in the sample.

In a preferred embodiment, the expression may be measured by measuring the level of mRNA.

Methods for measuring the level of mRNA are well known in the art. For example the nucleic acid contained in the samples (e.g., isolated cancer cells prepared from the patient, like those included in biopsies) is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). In a preferred embodiment, the expression of the CCL2, Cyr61, IL6, IGFBP7, CCL8, PAI1, OPG, MMP2 and UPAR genes is measured by RT-PCR, preferably quantitative or semi-quantitative RT-PCR, even more preferably real-time quantitative or semi-quantitative RT-PCR.

Other methods of amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above amplification and detection method may be assembled as a kit. Such a kit includes consensus primers and molecular probes. A preferred kit also includes the components necessary to determine if amplification has occurred. The kit may also include, for example, PCR buffers and enzymes; positive control sequences, reaction control primers; and instructions for amplifying and detecting the specific sequences.

In a particular embodiment, the methods of the invention comprise contacting the cancer cells of the biological sample with a binding partner capable of selectively interacting with the target protein, i.e. CCL2, Cyr61, IL6, IGFBP7, CCL8, PAI1, OPG, MMP2 or UPAR protein present in the biological sample. The binding partner may be an antibody that may be polyclonal or monoclonal, preferably monoclonal. In another embodiment, the binding partner may be an aptamer.

Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention or a fragment thereof can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985).

Alternatively, techniques described for the production of single chain antibodies (see e.g. U.S. Pat. No. 4,946,778) can be adapted to produce anti-CCL2 or anti-Cyr61 single chain antibodies. Antibodies useful in practicing the present invention also include anti-CCL2 fragments and anti-Cyr61 fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to CCL2 or Cyr61. For example, phage display of antibodies may be used. In such a method, single-chain Fv (scFv) or Fab fragments are expressed on the surface of a suitable bacteriophage, e. g., M13. Briefly, spleen cells of a suitable host, e. g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e. g., bacteria, the phage displays the antibody fragment. Phage display of antibodies may also be provided by combinatorial methods known to those skilled in the art. Antibody fragments displayed by a phage may then be used as part of an immunoassay.

In another embodiment, the binding partner may be an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. 1997. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consist of conformationally constrained antibody variable regions displayed by a platform protein, such as E. coli Thioredoxin A, that are selected from combinatorial libraries by two hybrid methods (Colas et al., 1996).

The binding partners of the invention, such as antibodies or aptamers, may be labelled with a detectable molecule or substance, such as a fluorescent molecule, a radioactive molecule or any others labels known in the art. Labels are known in the art that generally provide (either directly or indirectly) a signal.

As used herein, the term “labelled”, with regard to the antibody, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be labelled with a radioactive molecule by any method known in the art. For example radioactive molecules include but are not limited radioactive atom for scintigraphic studies such as I123, I124, In111, Re186, Re188.

The aforementioned assays generally involve the binding of the binding partner (ie. antibody or aptamer) to a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

The gene expression level of CCL2 or Cyr61 protein in cancer cells may be measured by using standard immunodiagnostic techniques, including immunoassays such as competition, direct reaction, or sandwich type assays. In such embodiments, cancers cells are purified from the isolated biological sample. Such assays include, but are not limited to, agglutination tests; enzyme-labelled and mediated immunoassays, such as ELISAs; biotin/avidin type assays; radioimmunoas says; immunoelectrophoresis; immunoprecipitation.

More particularly, an ELISA method can be used, wherein the wells of a microtiter plate are coated with a set of antibodies against the target (ie CCL2 or Cyr61). The cancer cells of the biological sample that are suspected of containing CCL2 or Cyr61, are then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

### FIGURE LEGENDS

FIG. 1

Melanoma Cells Develop a Senescence Associated Secretome.

(a) 501mel cells were seeded on the filter of the upper compartment and supernatants from control (CM-siC) or senescent (CM-siMi) 501mel cells were added to the lower compartment of Boyden chambers. Cells that had migrated to the underside of the upper compartment were stained 24 hrs later with crystal violet and the number of nuclei was counted using NIH-imageJ analysis software. Values represent mean+SD of three independent experiments. Representative images are shown. (b) The chemotactic effect of the conditioned media from 501mel cells exposed to fotemustine, temozolomide or H2O2 as mentioned above was tested on naive 501mel melanoma cells in Boyden chambers. The number of nuclei was counted using NIH-imageJ analysis software. Forty-eight hours after transfection, the medium was changed by DMEM 1% serum for additional 48 hrs. Drug-free conditioned media were next used in chemotaxis experiments with naive 501mel cells. Cells that had migrated were stained 24 hrs later with crystal violet and counted. Values represent mean+SD of two independent experiments. Representative images are shown.

FIG. 2

CCL2 is a Major Determinant of the PNAS.

(a) RNAs were harvested from control (siC) or MITF (siMi) siRNA for 96 hrs and were assayed by qRT-PCR for transcripts indicated on the figure. Transcript levels are represented relative to those found in control-transfected cells as mean+SD. The results in the graph are expressed as log 2 values. (b) ELISA test of CCL2 level in the conditioned media of control (CM-siC) or senescent (CM-siMi) 501mel melanoma cells. Data are represented as mean+SD. (c) The chemotactic effect of recombinant CCL2 (100 ng/ml) with or without anti-human CCL2 neutralizing antibody (100 μg/ml) was tested on naive 501mel melanoma cells in Boyden chambers and the number of nuclei was counted using NIH-imageJ analysis software. The average values from three experiments+SD are shown. Representative images are shown. (d) 501mel cells were left untreated (CM-C) or were exposed to fotemustine (FMT, 40 mM), temozolomide (TMZ, 900 nM) or H2O2 (100 mM) for 96 hrs. CCL2 secretion level was analyzed by ELISA. Values are expressed as mean+SD. (e) The chemotactic effect of control (CM-siC) or senescent (CM-siMi) cells, supplemented or not with anti-human CCL2 neutralizing antibody (100 μg/ml), was assessed on naive 501mel cells. The number of nuclei was counted using NIH-imageJ analysis software. Values are expressed as mean+SD. Representative images are shown.

FIG. 3

Stimulation of NF-kB Activity in Senescent Cells.

NF-kB luciferase promoter activity of 501mel cells transfected with MITF siRNA or exposed to fotemustine (FMT, 40 mM), temozolomide (TMZ, 900 nM) or H2O2 (100 mM) in presence or absence of sulfasalazine (500 μM). Results are expressed as percent (%)+SD of the luciferase activity from the control condition.

FIG. 4

NF-kB Drives CCL2 Secretion and Pro-Invasive Properties of the PNAS.

(a) mRNA levels of MITF, IKK2 and CCL2 in 501mel cells transfected with siC or siMi with or without siIKK2. Bars represent the mean+SD of three independent experiments performed in triplicate. (b) ELISA test of CCL2 secretion level in the culture medium of 501mel cell treated transfected with siC or siMi with or without siIKK2 or exposed to sulfasalazine. Values are expressed as mean+SD. (c) The chemotactic effect of the conditioned medium of melanoma cell treated as above. The number of nuclei was counted using NIH-imageJ analysis software. Values are expressed as mean+SD. Representative images are shown. (d) 501mel cells were transfected with siC or siMi. When indicated, cells were co-knockdown for IKK2 (siIKK2) or exposed to the NF-kB inhibitor sulfasalazine (sulfa, 500 μM) or to TNFα (10 ng/ml) for the 6 last hrs. Ninety-six hours later, the luciferase activity was assessed and normalized to the β-galactosidase activity. Results are expressed as percent (%)+SD of the luciferase activity from the control condition.

FIG. 5

The PARP-1/NF-kB Axis Plays a Key Role in the Deleterious Effect of the Secretome.

(a) NF-kB luciferase promoter activity of 501mel cells transfected with MITF siRNA or exposed to fotemustine (FMT, 40 mM), temozolomide (TMZ, 900 nM) or H2O2 (100 mM) in presence or absence of 3-AB (20 mM). Results are expressed as percent (%)+SD of the luciferase activity from the control condition. (b) CCL2 secretion level was analyzed by ELISA in the conditioned media of 501mel cells transfected with siC or siMi and exposed to the PARP-1 inhibitor 3-aminobenzamide (3-AB, 20 mM) or to the NF-kB inhibitor sulfasalazine (Sulfa, 500 ↑M) for 96 hrs. Values are expressed as mean+SD. (c) 501mel cells were transfected with siC or siMi and exposed when indicated to sulfasalazine (500 μM) or 3-AB (20 mM). Forty-eight hours after transfection and inhibitor exposure, the medium was changed by DMEM 1% serum for additional 48 hrs. Drug-free conditioned media were next used in chemotaxis experiments with naive 501mel cells. Cells that had migrated were stained 24 hrs later with crystal violet and counted. Values represent mean+SD of two independent experiments.

FIG. 6

The PARP-1/NF-kB Axis is Also Engaged in the PNAS of Non-Melanoma Cancer Cells.

(a) Conditioned media of control or senescent MCF7 (H2O2 treated) were added to the lower compartment of Boyden chambers to assess their chemotactic activity. Cells that had migrated to the underside of the upper compartment were stained 24 hrs later with crystal violet and the number of nuclei was counted using NIH-imageJ analysis software. Values represent mean+SD of three independent experiments. (b) ELISA test of CCL2 level in the conditioned media of control MCF7 or MCF7 incubated with H2O2 (100 mM) and when indicated with sulfasalazine (500 μM) or 3-AB (20 mM). Data are represented as mean+SD.

FIG. 7

Graphical Abstract of the Molecular Determinants of PNAS Formation and Deleterious Effects.

Chemotherapeutic drugs or oxidative stress induce DNA damages through stimulation of the PARP-1/ATM axis and senescence entry. The senescence phenotype is associated with the development of a secretome that enhances invasion of melanoma cells that might have escaped the process of senescence. Blocking PARP-1, ATM or NF-kB prevents the deleterious pro-invasive properties of the senescence state.

### EXAMPLE 1

Materials and Methods

Cell Cultures and Reagent

501mel human melanoma cells and MCF7 human breast cancer cells were grown in DMEM supplemented with 7% FBS at 37° C. in a humidified atmosphere containing 5% CO2. Lipofectamine™ RNAiMAX and opti-MEM medium were purchased from Invitrogen (San Diego, Calif., USA). TNFa and recombinant human CCL2 were from PeproTech Inc (Rocky Hill, N.J.), sulfasalazine and 3-AB from Sigma Chem. Co (St Louis, Mo.), mouse anti-CCL2 from R&D Systems and KU55933 from Calbiochem. The senescence b-galactosidase staining kit from Cell Signaling Technology (Beverly, Mass., USA) was used to histochemically detect b-galactosidase activity at pH6 as previously reported (Giuliano et al. 2010a). The percentage of means and standard deviations were derived from counting 100 cells in duplicate plates after 96 hrs.

Transient Transfection of siRNA

Briefly, a single pulse of 50 nM of siRNA was administrated to the cells at 50% confluency by transfection with 5 ml Lipofectamine™ RNAiMAX in opti-MEM medium (Invitrogen, San Diego, Calif., USA). Control (siC) and MITF (siMi) siRNAs were previously described (Larribere et al. 2005). When indicated, IKK2 was co-knocked down (IKK2-Stealth-RNAi, Invitrogen).

Invasion Assay

Cell invasion was assessed using a modified Boyden chamber assay with 8-mm pore filter inserts for 24-well plates (BD Bioscience). 501mel cells were seeded on the upper chamber of matrigel-coated trans-well and chemoattractants (conditioned media or recombinant molecules) placed into the lower chamber. Twenty-four hours later, cells adherent to the underside of the filters were fixed with 4% PFA, stained with 0.4% crystal violet and five random fields at ×20 magnification were counted. Results represent the average of triplicate samples from three independent experiments.

Western Blot Assays

Western blots were carried out as previously described (Hilmi et al. 2008). Briefly, cell lysates (30 μg) were separated by SDS-PAGE, transferred on to a PVDF membrane and then exposed to the appropriate antibodies, anti-MITF (Abcam), anti-ERK2 (D-2 Santa Cruz biotechnology), anti-p53 (DO-1, Santa Cruz biotechnology), anti-PARP (Santa Cruz biotechnology), anti-IKK2 and anti-PARP (Cell Signaling Technology Inc.). Horseradish peroxidase-conjugated anti-rabbit or anti-mouse antibodies were from Dakopatts (Glostrup, Denmark). Proteins were visualized with the ECL system (Amersham). The western blots shown are representative of at least 3 independent experiments.

mRNA Preparation, Real-Time/Quantitative PCR

mRNA isolation was performed with Trizol (Invitrogen), according to standard procedure. QRT-PCR was carried out with SYBR® Green I (Eurogentec, Seraing, Belgium) and Multiscribe Reverse Transcriptase (Applied Biosystems) and monitored by an ABI Prism 7900 Sequence Detection System (Applied Biosystems, Foster City, Calif.). Detection of SB34 gene was used to normalize the results. Primer sequences for each cDNA were designed using either Primer Express Software (Applied Biosystems) or qPrimer depot (http://primerdepot.nci.nih.gov) and are available upon request.

Luciferase Reporter Assays

501mel melanoma cells were transiently transfected as previously described using the lipofectamine reagent (Invitrogen) (Bertolotto et al. 1998). Briefly, cells were transiently transfected with 0.3 μg of NF-kB reporter construct and 0.05 μg pCMVbGal to control the variability in transfection efficiency. The transfection medium was changed 6 hours later and when indicated, cells were transfected as described above with 50 nM of siC or siMi and/or siIKK2 or were exposed to drugs. Cells were assayed for luciferase and b-galactosidase activities 48 hours later. Transfections were repeated at least three times.

### Immunofluorescence Studies

Melanoma cells seeded on glass coverslips in 12-well dishes, were transfected with siC or siMi. When indicated cells were exposed to TNFa (10 ng/ml). Ninety-six hours later, cells were fixed and permeabilized as previously described (Khaled et al. 2003) before being exposed to anti-MITF (Abcam), anti-p65/RelA (Santa Cruz biotechnology), 53BP1 (Bethyl Laboratories) and E-cadherin (BD Transduction Laboratories) antibodies. Cells were washed thrice with PBS, and then incubated for 1 hour with 1:1000 dilution anti-mouse or anti-rabbit Alexa Fluor 488 or Alexa Fluor 594 labeled secondary antibody (Invitrogen, San Diego, Calif., USA) and mounted using Gel/Mount (Biomeda corp., Foster City Calif.). Immunofluorescences were examined and photographed with a Zeiss Axiophot microscope equipped with epifluorescence illumination.

ELISA

CCL2 level in the supernatant of the different melanoma cell lines, in that of MCF7 cells or in that of 501mel cells transfected with siC or siMi for 96 hrs, in presence or absence of drugs, was quantified by ELISA (R&D systems). Results from two independent experiments were normalized to cell number and expressed as ng/ml/10⁶ cells.

EMSA

Electrophoretic mobility shift assay (EMSA) for NF-kB was performed as described previously (Imbert et al. 1996). Briefly, nuclear extracts prepared from 501mel cells transfected with siC or siMi or treated with TNFa were incubated with ³²P-end-labeled synthetic double-stranded oligonucleotide containing the κB binding site of the immunoglobulin promoter for 20 min at 37° C., and the DNA-protein complex formed was separated from free oligonucleotide on 5% nondenaturating polyacrylamide gels in 0.5× Tris-Borate EDTA.

Results

Senescent Melanoma Cells Develop a Secretome with Pro-Invasive Properties

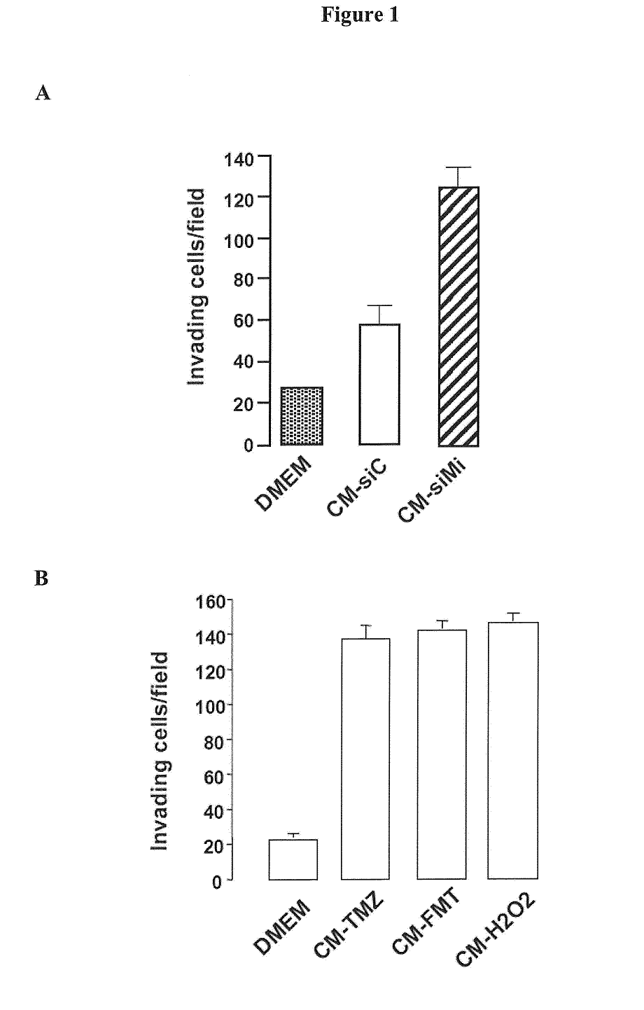

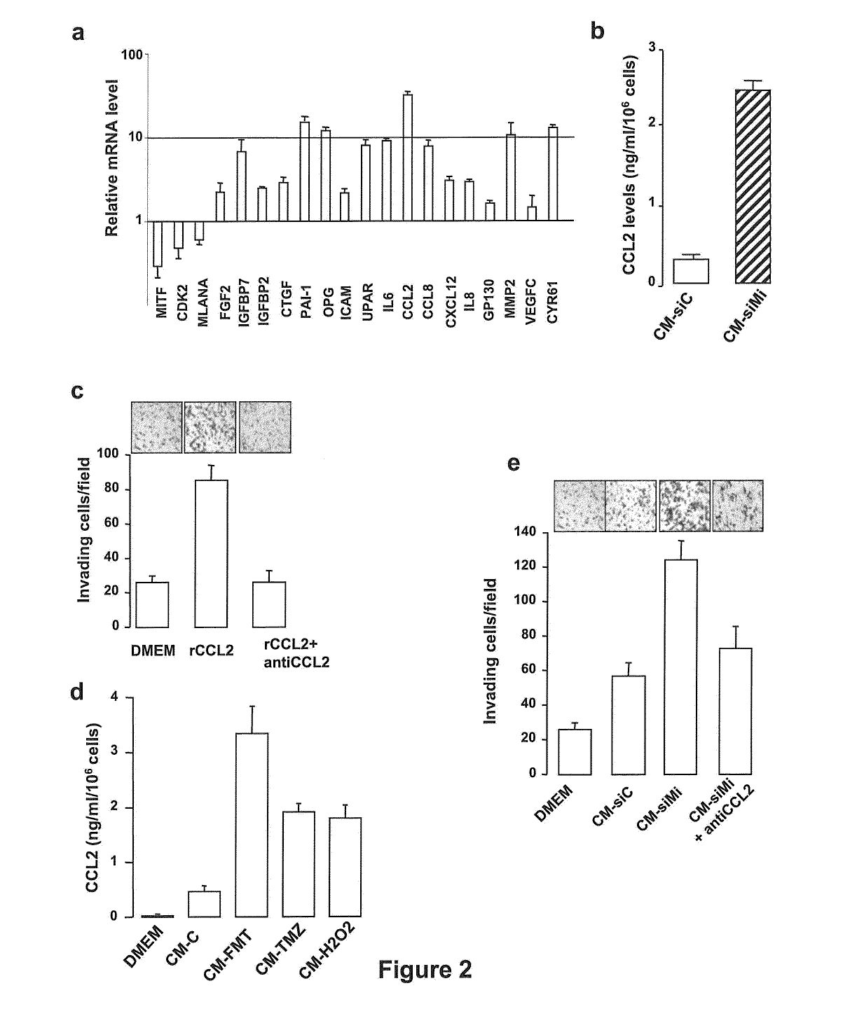

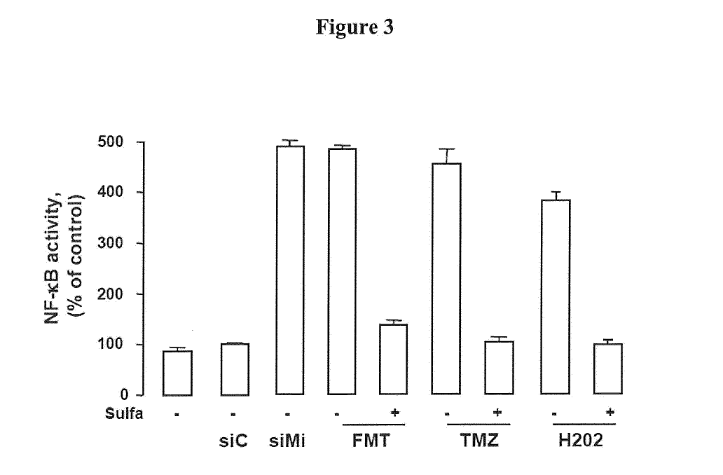

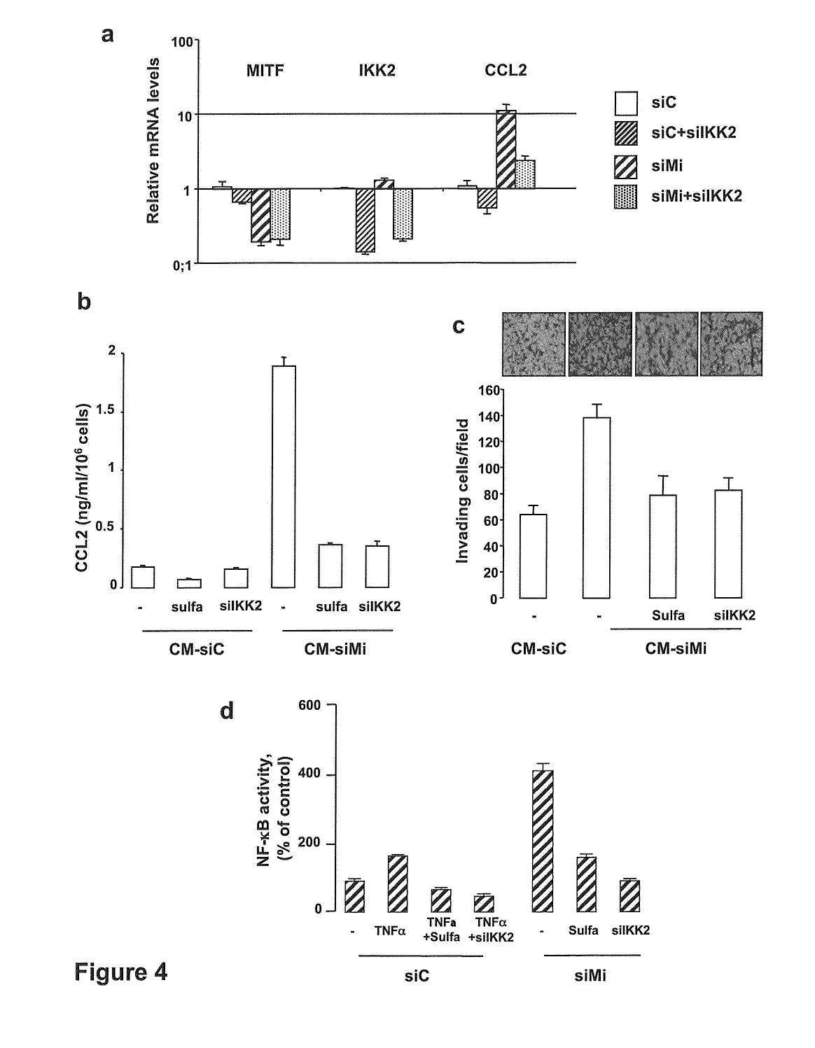

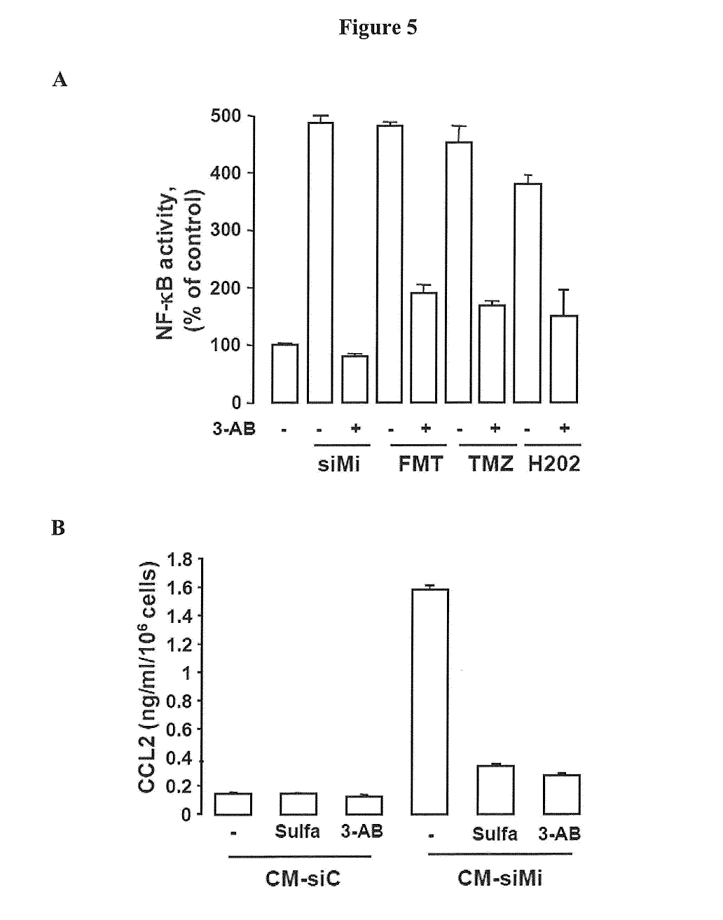

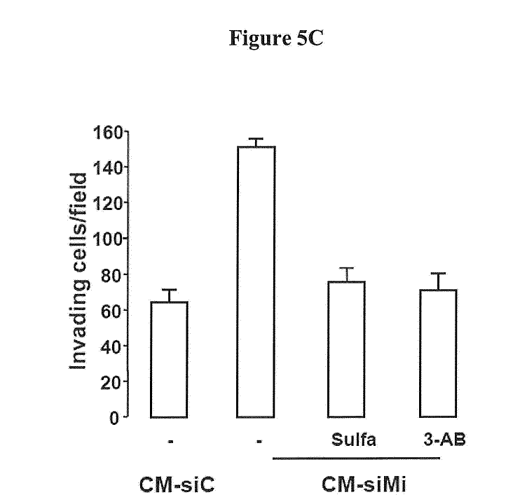

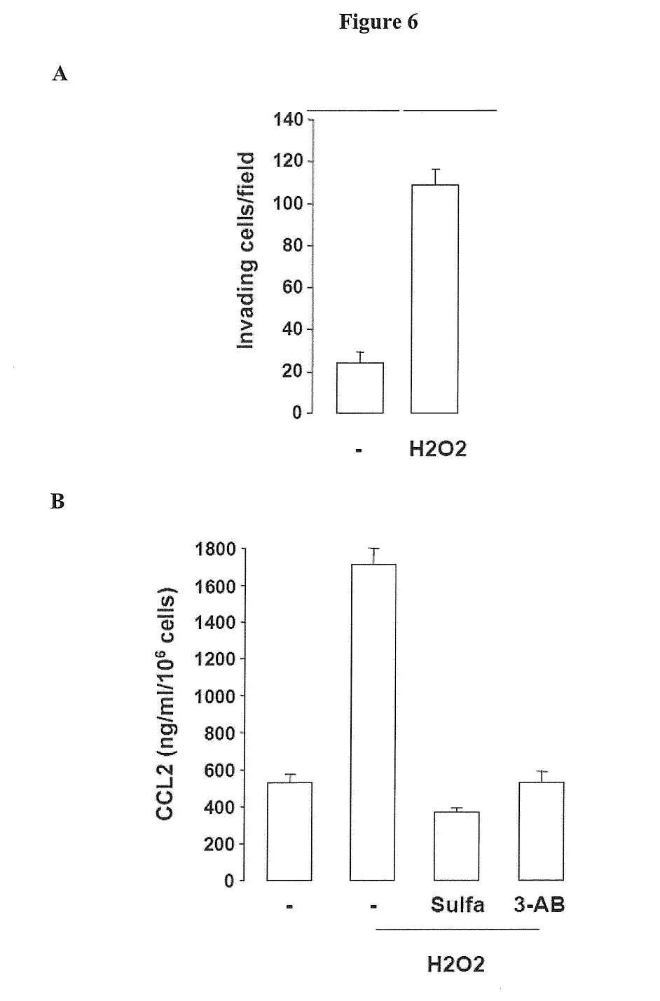

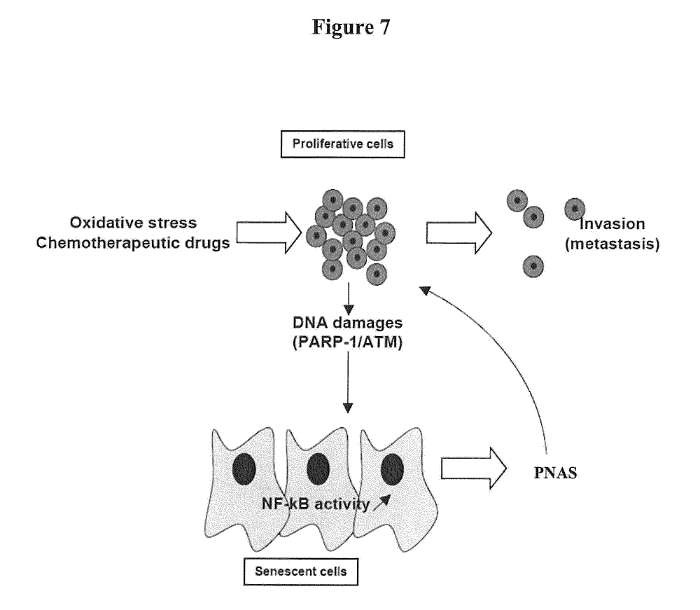

As previously shown, MITF suppression by specific siRNA led to melanoma cell senescence characterized by the increased expression in both p27^{KIPI} and p53 (data not shown) and senescence associated-b-galactosidase reactivity at pH6 (SA-b-Gal) (data not shown). To determine whether senescent melanoma cells were able to produce an active secretome, naive 501mel melanoma cells were incubated with the conditioned medium (CM) from 501mel melanoma cells transfected with control or MITF siRNA. When exposed to the conditioned medium from senescent melanoma cells, 501mel melanoma cells exhibited a decreased expression of E cadherin (data not shown), a marker of epithelial-mesenchymal transition that was associated with tumor formation and increased aggressiveness. Experiments using matrigel-coated chambers revealed that the secretome of senescent cells favored invasion of naive 501mel melanoma cells (FIG. 1A). Altogether, MITF depletion promotes senescence of melanoma cells and triggers the production of a senescence-associated secretory phenotype with pro-invasive properties.

We next wished to investigate whether different stimuli, such as oxidative stress, a common inducer of premature senescence, or temozolomide a chemotherapeutic drugs that has been shown to trigger melanoma cell senescence, might also induce a secretome. This hypothesis was strengthened by the recent observations that H2O2 or temozolomide, one of the first-line alkylating agent used in melanoma therapy, decreased the expression of MITF (Mhaidat et al. 2007; Liu et al. 2009). Here, we confirmed that melanoma cells exposed to H2O2 or temozolomide underwent premature senescence, characterized by the flattened and enlarged morphology and SA-b-Gal reactivity at pH6 (data not shown). Noteworthy, we extended these findings to fotemustine, another drug for treating metastatic melanomas. In this context, oxidative stress and chemotherapeutic drugs elicited formation of a senescence-associated secretory phenotype that enhanced invasion of melanoma cells (FIG. 1B).

CCL2 is a Major Actor of the Pro-Invasive Function of the Secretome

Data sets from DNA microarray experiments, in which gene profile between control or MITF-silenced melanoma cells, were investigated revealed an up-regulation of several secreted factors (see Table 1 at the end of the description). qRT-PCR experiments were also performed on a panel of cytokines previously reported in the secretome of human fibroblasts and epithelial cells (Coppe et al. 2008). In addition to the inhibition of known MITF target genes that validates these experiments, we identify the repertoire of the secretory profile in senescent melanoma cells, demonstrating the presence of growth factors (CTGF, VEGF), proteases (PAI-1, MMP2), interleukins (IL6) and chemokines (CCL2, CCL8) (FIG. 2a). Among them, CCL2 appeared as one of the most up-regulated factors. ELISA tests confirmed the increased secretion of CCL2 in the conditioned medium of senescent melanoma cells relative to control cells (FIG. 2b).

Interestingly, the effect of MITF silencing on increased CCL2 mRNA and secretion was counteracted by restoring MITF expression, thereby ruling out a non-specific effect of siRNA (data not shown). Experiments in Boyden chamber showed that CCL2 stimulated the invasive capability of naive 501mel melanoma cells while an anti-CCL2 neutralizing antibody almost completely abolished this function (FIG. 2c). Interestingly, CCL2 secretion was also strongly increased in the secretome of melanoma cells exposed to oxidative stress or chemotherapeutic drugs (FIG. 2d). Importantly, an anti-CCL2 neutralizing antibody dramatically reduced the pro-invasive effects of the secretome (FIG. 2e). In all, these results demonstrate that melanoma cells undergoing senescence develop a secretome, with pro-invasive properties, in which the chemokine CCL2 is a critical factor.

The NF-kB Signaling Pathway Controls Senescence-Associated Secretome Formation

It is noteworthy that the secretome comprises several pro-inflammatory factors prompting us to envision an involvement of the NF-kB signaling pathway in its formation. NF-kB is present as a latent, inactive cytoplasmic form while activated NF-κB is translocated into the nucleus where it binds to specific kB enhancer elements in the promoter of its target genes. First, we investigated by immunofluorescence experiments the cellular localization of the p65/RELA subunit of NF-kB. In control condition, p65 mainly localized to the cytoplasm while in response to TNFa, a known stimulator of NF-kB activity, p65 relocalized to the nucleus (data not shown). Interestingly, a massive nuclear accumulation of p65/RELA could be observed in melanoma cells rendered senescent by MITF depletion (data not shown). It is worth mentioning that the level of nuclear NF-kB was more important than that caused by TNFa. Consistently, EMSA revealed a stronger NF-kB binding in MITF-depleted 501mel cells compared to control or TNFa-treated cells (data not shown). A cell based luciferase reporter assay confirmed that MITF depletion increased the transcriptional activity of NF-kB (FIG. 3). Furthermore, oxidative stress and the chemotherapeutic drugs also stimulated in a similar range the transcriptional activity of NF-kB. Hence, the senescence like phenotype is associated with stimulation of the transcriptional activity of NF-kB.

NF-kB Controls CCL2 Secretion and the Invasive Function of the Secretome

Having shown that senescent melanoma cells produce a secretome and displayed an increased NF-kB activity, we next thought to determine the role of NF-kB in its formation. qRT-PCR (FIG. 4a) and ELISA (FIG. 4b) showed a stimulation of both CCL2 mRNA level and CCL2 secretion respectively in MITF-silenced cells, relative to control cells, that were impaired by genetic or pharmacological inhibition of NF-kB. Furthermore, blocking NF-kB prevented the pro-invasive effect of the secretome (FIG. 4c). NF-kB transcriptional activity was indeed up-regulated in TNFa exposed and much more in MITF-silenced cells compared to control 501mel melanoma cells (FIG. 4d). Inhibition of the NF-kB signaling pathway through pharmacologic (sulfasalazine) or genetic (IKK2 siRNA and dominant negative forms of IKK1 and IKK2) approaches prevented activation of the NF-kB reporter gene by siMITF or TNFa (FIG. 4d). Putting together, these observations demonstrate that melanoma cells undergoing senescence exhibited an enhanced NF-kB activity, that controls CCL2 production and its pro-invasive effect.

Secretome Formation Relies on DNA Damage Signaling-Induced NF-kB Activation

The genotoxic sensor poly(ADP-ribose)-polymerase-1 (PARP-1) has been involved in the activation of NF-kB (Stilmann et al. 2009). Interestingly, we previously demonstrated that MITF-silencing triggers DNA damage (Giuliano et al. 2010a). Kinetic studies revealed by western blot no modification of PARP-1 expression upon MITF-silencing (data not shown). However, increased poly-(ADP rybosyl)ation was observed in MITF-silenced melanoma cells and in cells exposed to H2O2 as previously shown (Kolthur-Seetharam et al. 2006) (data not shown). To screen the involvement of PARP-1 in NF-kB activity and in secretome formation, we used the PARP inhibitor 3-AB. In presence of 3-AB or sulfasalazine, MITF-silencing was no longer able to relocate NF-kB to the nucleus (data not shown) and to stimulate the NF-kB transcriptional activity (FIG. 5a). Blockade of the NF-kB activity by 3-AB was comparable to that elicited by sulfasalazine in agreement with these factors functioning in the same pathway (data not shown). Western blot analysis of the corresponding cell extracts revealed that MITF silencing increased IKK2 and p53, which affirmed engagement of the DNA-damages/NF-kB axis. IKK2 up-regulation mediated by MITF silencing was abrogated by sulfasalazine (data not shown). Contrastingly, p53 up-regulation was modestly affected by sulfasalazine but almost completely abrogated by 3-AB.

Similar to MITF depletion, focal staining of 53BP1 was also observed when melanoma cells were exposed to oxidative stress and chemotherapeutic drugs (data not shown), indicating the presence of DNA damages. Consistent with our hypothesis, the oxidative stress and chemotherapeutic drugs also stimulated the transcriptional activity of NF-kB that was prevented by addition of 3-AB (FIG. 5a). Additionally, blocking PARP-1 or NF-kB signaling pathways prevented to the same extent the secretion of CCL2 (FIG. 5b) and invasion as illustrated by results from Boyden chamber experiments (FIG. 5c). Noteworthy, to avoid the presence of the inhibitors in the conditioned media, which could affect by themselves the migrative properties of naive melanoma cells, the media were changed before being used in chemotaxis experiments. Together, in melanoma cells, the secretome is governed by a PARP-1 and NF-kB signaling cascade.

PNAS in Non-Melanoma Cells

We next wondered if a similar PARP-1 and NF-kB-assiocated secretome (PNAS) could be observed in non-melanoma cells that have entered a program of senescence. To this aim, we used the model of the MCF7 human breast cancer cells that underwent senescence upon H2O2 as illustrated by morphological changes, senescence-associated b-galactosidase reactivity (data not shown) and increased expression in the senescence markers p53 and p21 (data not shown). Senescence like phenotypes of MCF7 in response to H2O2 was associated with DNA damage induction as indicated by gH2AX labeling and stimulation of NF-kB activity shown by the nuclear localization of p65/RELA (data not shown). In agreement with the production of a pro-inflammatory secretome, the supernatant of senescent MCF7 enhanced the invasion of naïve MCF7 cells (FIG. 6a). Although CCL2 was not expected to be the major component of MCF7's secretome, CCL2 measurement was used as a readout of its formation. In this context, senescence of MCF7 cells resulted in CCL2 secretion, that was impaired when MCF7 cells were incubated with 3-AB or sulfasalazine (FIG. 6b). Together, our result reveal that PNAS during cellular senescence is a common mechanism that relies on activation of the the DNA damages/NF-kB signaling cascade in both melanoma and non-melanoma cell lines.

TABLE 1
cDNA microarray analysis of gene expression changes in MITF-depleted
compared to control human melanoma cells

| Gene Symbol | GenBank ID | Fold^{a} | ±SD |
|---|---|---|---|
| MLANA^{b} | 2315 | −3.396 | 0.00469335 |
| DCT^{b} | 1638 | −2.926 | 0.05056096 |
| GPR143^{b} | 4935 | −2.576 | 0.01523909 |
| CDK2^{b} | 12566 | −2.480 | 0.16978766 |
| MITF^{b} | 17342 | −2.451 | 0.23248394 |
| EDNRB^{b} | 1310 | −2.006 | 0.0601292 |
| TYR^{b} | 7299 | −1.723 | 0.31832707 |
| TYMS | 7298 | −1.422 | 0.08074477 |
| TLR4 | 7099 | −0.933 | 0.03433735 |
| GAS1 | 2619 | −0.811 | 0.12216614 |
| CSF1 | 1435 | −0.371 | 0.04441918 |
| MMP2 | 17390 | −0.363 | 0.05642465 |
| MMP19 | 4327 | −0.343 | 0.09860566 |
| IL23A | 51561 | −0.269 | 0.12139847 |
| PLCD3 | 113026 | −0.258 | 0.05592975 |
| IL1B | 3553 | −0.244 | 0.03340169 |
| CCR2 | 729230 | −0.236 | 0.19877822 |
| CCL20 | 6364 | −0.243 | 0.00974077 |
| IL12B | 3563 | −0.213 | 0.07021685 |
| PLA2G2A | 5320 | −0.213 | 0.04219609 |
| MMP24 | 10893 | −0.213 | 0.12310318 |
| MMP3 | 4314 | −0.195 | 0.09721145 |
| IFNG | 3458 | −0.188 | 0.11872011 |
| CCL23 | 6368 | −0.185 | 0.08916862 |
| TK1 | 21877 | −0.184 | 0.05639935 |
| MMP25 | 64386 | −0.175 | 0.03900126 |
| CXCL2 | 20310 | −0.149 | 0.03691421 |
| IL25 | 64806 | −0.142 | 0.04799052 |
| CCL13 | 6357 | −0.141 | 0.04416873 |
| MMP27 | 64066 | −0.136 | 0.03272653 |
| CXCL13 | 10563 | −0.135 | 0.09607781 |
| LTA | 4049 | −0.134 | 0.00088346 |
| CCL1 | 6346 | −0.129 | 0.02817336 |
| NOS2A | 4843 | −0.128 | 0.00096244 |
| TNC | 3371 | −0.125 | 0.10351928 |
| CSF3 | 1440 | −0.121 | 0.03524244 |
| CTSW | 1521 | −0.118 | 0.01530317 |
| IL20 | 50604 | −0.114 | 0.04449025 |
| CTSD | 1509 | −0.108 | 0.11152328 |
| PTGS1 | 5742 | −0.106 | 0.08950215 |
| MMP28 | 79148 | −0.106 | 0.10373071 |
| COL4A6 | 1288 | −0.103 | 0.04429363 |
| MMP12 | 17381 | −0.103 | 0.0940134 |
| NRG1 | 3084 | −0.102 | 0.00229363 |
| IL26 | 55801 | −0.091 | 0.12030261 |
| CCL21 | 6366 | −0.089 | 0.0462948 |
| CDKN1A | 1026 | −0.087 | 0.00241119 |
| CXCL6 | 6373 | −0.079 | 0.05563248 |
| CXCL16 | 58191 | −0.079 | 0.04920185 |
| TNF | 7124 | −0.076 | 0.10599916 |
| CCL15 | 6359 | −0.074 | 0.1840475 |
| IL22 | 50616 | −0.069 | 0.04443437 |
| CTSF | 8722 | −0.064 | 0.17873298 |
| CCL17 | 6361 | −0.060 | 0.02860028 |
| CCL25 | 6370 | −0.060 | 0.03206517 |
| CXCL3 | 2921 | −0.056 | 0.05773861 |
| STC1 | 6781 | −0.055 | 0.08652515 |
| ICAM2 | 3384 | −0.050 | 0.034037 |
| BMP4 | 652 | −0.047 | 0.16797378 |
| IL-8 | 3578 | −0.04 | 0.12030261 |
| MMP1 | 4312 | −0.039 | 0.03421012 |
| IL10 | 3586 | −0.039 | 0.28276301 |
| CXCL9 | 4283 | −0.031 | 0.13624919 |
| MIA | 8190 | −0.028 | 0.13349621 |
| MMP13 | 4322 | −0.027 | 0.03293029 |
| MMP25 | 64386 | −0.024 | 0.07126598 |
| CXCL5 | 6374 | −0.023 | 0.05766034 |
| ICAM4 | 3386 | −0.021 | 0.0602564 |
| ICAM5 | 7087 | −0.021 | 0.01765933 |
| CTSG | 1511 | −0.019 | 0.04826551 |
| GADD45G | 10912 | −0.018 | 0.01332963 |
| CCL19 | 6363 | −0.017 | 0.02546197 |
| CTSG | 1511 | −0.017 | 0.03127073 |
| CSF2 | 1437 | −0.016 | 0.19010607 |
| CDH2 | 1000 | −0.013 | 0.14005396 |
| PTGDS | 5730 | −0.013 | 0.03829708 |
| CXCL12 | 6387 | −0.007 | 0.0534576 |
| CXCL2 | 2920 | −0.007 | 0.01285506 |
| CCL16 | 6360 | −0.004 | 0.07659257 |
| LTB | 4050 | −0.003 | 0.06626507 |
| IL18 | 3606 | 0.001 | 0.08638869 |
| CCL8 | 6355 | 0.001 | 0.009756 |
| RELB | 5971 | 0.009 | 0.072504 |
| CCL22 | 6367 | 0.010 | 0.05544205 |
| CTSO | 1519 | 0.011 | 0.08053704 |
| CTSH | 1512 | 0.012 | 0.03064311 |
| CCL24 | 6369 | 0.013 | 0.10769452 |
| VCAM1 | 7412 | 0.051 | 0.07163438 |
| IGFBP5 | 3488 | 0.052 | 0.10329463 |
| CTSK | 1513 | 0.053 | 0.01863409 |
| IGFBP4 | 3487 | 0.059 | 0.03129747 |
| IL9 | 3578 | 0.063 | 0.10903487 |
| PLAUR | 5329 | 0.063 | 0.05902043 |
| IL21 | 59067 | 0.074 | 0.05101306 |
| FN1 | 2335 | 0.074 | 0.07163438 |
| MMP9-8732 | 4318 | 0.079 | 0.05144167 |
| MMP10 | 4319 | 0.092 | 0.04333998 |
| TGFB3 | 25717 | 0.098 | 0.05451782 |
| IGFBP1 | 3484 | 0.102 | 0.06091146 |
| CXCL1 | 2919 | 0.106 | 0.05529325 |
| MMP11 | 4320 | 0.109 | 0.0335571 |
| CCL26 | 10344 | 0.109 | 0.07018633 |
| MMP7 | 4316 | 0.109 | 0.11580969 |
| TNFRSF9 | 3604 | 0.118 | 0.08760524 |
| IL29 | 282618 | 0.121 | 0.01351721 |
| IFNA21 | 3452 | 0.123 | 0.1656126 |
| CCL28 | 56477 | 0.135 | 0.01215404 |
| ICAM3 | 3385 | 0.154 | 0.03228226 |
| CCL7 | 6354 | 0.154 | 0.05752267 |
| CXCL11 | 6373 | 0.156 | 0.02812627 |
| SERPINE2 | 5270 | 0.157 | 0.10468166 |
| FLT3LG | 2323 | 0.159 | 0.04443621 |
| BTC | 685 | 0.160 | 0.00355782 |
| CSF3R | 1441 | 0.175 | 0.07321103 |
| CTSC | 1075 | 0.182 | 0.02526494 |
| VIM | 7431 | 0.187 | 0.03228226 |
| JUNB7 | 3726 | 0.187 | 0.01970141 |
| HSPA6 | 3310 | 0.194 | 0.13625277 |
| LSP1 | 4046 | 0.195 | 0.00634355 |
| FGF12 | 2257 | 0.207 | 0.01286341 |
| COL9A2 | 1298 | 0.211 | 0.04611897 |
| IER3 | 8870 | 0.216 | 0.11462237 |
| FGF1 | 2246 | 0.222 | 0.13785331 |
| HDGF | 3068 | 0.235 | 0.23390925 |
| MST1 | 4485 | 0.235 | 0.08689901 |
| TNFRSF11B | 4982 | 0.238 | 0.01981589 |
| NFKB2 | 4791 | 0.246 | 0.13918396 |
| SERPINE1 | 5054 | 0.255 | 0.07002693 |
| AGT | 183 | 0.263 | 0.04780101 |
| MIF | 4282 | 0.278 | 0.03198065 |
| IL11 | 3589 | 0.296 | 0.08402775 |
| NGFR | 4808 | 0.307 | 0.0206672 |
| FGFR4 | 2264 | 0.309 | 0.02807424 |
| AGGF1 | 55109 | 0.312 | 0.00700683 |
| SMAD7 | 4092 | 0.331 | 0.11960035 |
| IL6 | 3569 | 0.367 | 0.07163438 |
| IGFBP6 | 3489 | 0.383 | 0.06735018 |
| IL27 | 246778 | 0.392 | 0.0212541 |
| VEGFC | 7424 | 0.413 | 0.01907472 |
| TRAF2 | 7186 | 0.416 | 0.10388079 |
| CCL4 | 6351 | 0.417 | 0.03471908 |
| SDC4 | 6385 | 0.427 | 0.02426495 |
| PDGFD | 80310 | 0.433 | 0.0571927 |
| COL2A1 | 1280 | 0.470 | 0.05351271 |
| BMP6 | 654 | 0.488 | 0.00993673 |
| TNFRSF1A | 7132 | 0.527 | 0.04277348 |
| SOD1 | 6647 | 0.529 | 0.04344671 |
| FAM14B | 122509 | 0.532 | 0.05758841 |
| CTSZ | 1522 | 0.547 | 0.05748764 |
| TRAF3IP1 | 26146 | 0.554 | 0.00586068 |
| BIRC2 | 329 | 0.563 | 0.02167024 |
| HDGF2 | 84717 | 0.621 | 0.00114578 |
| LAMB2 | 3913 | 0.622 | 0.01686728 |
| TIMP1 | 7076 | 0.637 | 0.02710985 |
| IGFBP3 | 3486 | 0.645 | 0.1682942 |
| IL16 | 3603 | 0.647 | 0.12020172 |
| IL24 | 11009 | 0.695 | 0.11939343 |
| PDGFB | 5155 | 0.695 | 0.11205684 |
| CTSB | 1508 | 0.699 | 0.14102819 |
| GADD45A | 1647 | 0.712 | 0.10124251 |
| SEMA4B | 10509 | 0.840 | 0.18886948 |
| ITGB1 | 16412 | 0.847 | 0.06665488 |
| PDE2A | 5138 | 1.133 | 0.02905928 |
| BDNF | 627 | 1.146 | 0.05858526 |
| CTGF | 1490 | 1.244 | 0.14782365 |
| IGFBP7 | 3490 | 1.282 | 0.15335056 |
| PTGS2 | 5743 | 1.285 | 0.07504039 |
| AMH | 268 | 1.336 | 0.06635736 |
| PLAT | 5327 | 1.430 | 0.26339494 |
| TNFRSF10B | 8795 | 1.457 | 0.03303672 |
| NQO1 | 1728 | 1.475 | 0.06055538 |
| CSK | 1445 | 1.488 | 0.0175709 |
| ITGB5 | 3693 | 1.520 | 0.09783993 |
| SEMA3B | 7869 | 1.530 | 0.03051046 |
| TNFRSF12A | 51330 | 1.631 | 0.12770329 |
| GADD45B | 4616 | 1.717 | 0.05688114 |
| IGFBP2 | 3485 | 1.841 | 0.09972546 |
| TGFBI | 7045 | 1.912 | 0.00266754 |
| COL9A3 | 1299 | 1.913 | 0.22407717 |
| PAM | 5066 | 2.003 | 0.03775165 |
| PLA2G4A | 5321 | 2.006 | 0.05901066 |
| TNFRSF21 | 27242 | 2.019 | 0.12051874 |
| IGFBP5 | 3488 | 2.021 | 0.0169905 |
| CXCL14 | 9547 | 2.832 | 0.01878546 |
| CEBPD | 1052 | 3.294 | 0.11188847 |
| CCL2 | 6347 | 3.326 | 0.0137319 |
| CYR61 | 3491 | 3.644 | 0.13448109 |

^{a}The ratio results (siMi/siC) are expressed as the mean +/− SD of log2-fold values. We pooled the raw data from two siC and two different MITF siRNA (siMi1 and siMi2)
^{b}represent known MITF target genes (Cheli et al, 2010).

### REFERENCES

Throughout this application, various references describe the state of the art to which the invention pertains. The disclosures of these references are hereby incorporated by reference into the present disclosure.
Acosta, J. C., O'Loghlen, A., Banito, A., Guijarro, M. V., Augert, A., Raguz, S., Fumagalli, M., Da Costa, M., Brown, C., Popov, N., Takatsu, Y., Melamed, J., d′Adda di Fagagna, F., Bernard, D., Hernando, E., and Gil, J. 2008. Chemokine signaling via the CXCR2 receptor reinforces senescence. Cell 133(6): 1006-1018.
Bavik, C., Coleman, I., Dean, J. P., Knudsen, B., Plymate, S., and Nelson, P. S. 2006. The gene expression program of prostate fibroblast senescence modulates neoplastic epithelial cell proliferation through paracrine mechanisms. Cancer Res 66(2): 794-802.
Bertolotto, C., Busca, R., Abbe, P., Bille, K., Aberdam, E., Ortonne, J. P., and Ballotti, R. 1998. Different cis-acting elements are involved in the regulation of TRP1 and TRP2 promoter activities by cyclic AMP: pivotal role of M boxes (GTCATGTGCT) and of microphthalmia. Mol Cell Biol 18(2): 694-702.
Cheli, Y., Ohanna, M., Ballotti, R., and Bertolotto, C. 2010. Fifteen-year quest for microphthalmia-associated transcription factor target genes. Pigment Cell Melanoma Res 23(1): 27-40.
Coppe, J. P., Patil, C. K., Rodier, F., Sun, Y., Munoz, D. P., Goldstein, J., Nelson, P. S., Desprez, P. Y., and Campisi, J. 2008. Senescence-associated secretory phenotypes reveal cell-nonautonomous functions of oncogenic RAS and the p53 tumor suppressor. PLoS Biol 6(12): 2853-2868.
d′Adda di Fagagna, F. 2008. Living on a break: cellular senescence as a DNA-damage response. Nat Rev Cancer 8(7): 512-522.
Dantzer, F., Ame, J. C., Schreiber, V., Nakamura, J., Menissier-de Murcia, J., and de Murcia, G. 2006. Poly(ADP-ribose) polymerase-1 activation during DNA damage and repair. Methods Enzymol 409: 493-510.
Davis, I. J., Kim, J. J., Ozsolak, F., Widlund, H. R., Rozenblatt-Rosen, O., Granter, S. R., Du, J., Fletcher, J. A., Denny, C. T., Lessnick, S. L., Linehan, W. M., Kung, A. L., and Fisher, D. E. 2006. Oncogenic MITF dysregulation in clear cell sarcoma: defining the MiT family of human cancers. Cancer Cell 9(6): 473-484.
Elledge, S. J. 1996. Cell cycle checkpoints: preventing an identity crisis. Science 274(5293): 1664-1672.
Garraway, L. A., Widlund, H. R., Rubin, M. A., Getz, G., Berger, A. J., Ramaswamy, S., Beroukhim, R., Milner, D. A., Granter, S. R., Du, J., Lee, C., Wagner, S. N., Li, C., Golub, T. R., Rimm, D. L., Meyerson, M. L., Fisher, D. E., and Sellers, W. R. 2005. Integrative genomic analyses identify MITF as a lineage survival oncogene amplified in malignant melanoma. Nature 436(7047): 117-122.
Giuliano, S., Cheli, Y., Ohanna, M., Bonet, C., Beuret, L., Bille, K., Loubat, A., Hofman, V., Hofman, P., Ponzio, G., Bahadoran, P., Ballotti, R., and Bertolotto, C. 2010a. MITF controls the DNA Damage Response and a lineage specific senescence program in melanomas Submitted.
Giuliano, S., Ohanna, M., Ballotti, R., and Bertolotto, C. 2010b. Advances in melanoma senescence and potential clinical application. Pigment Cell Melanoma Res In press.
Hartwell, L. H. and Kastan, M. B. 1994. Cell cycle control and cancer. Science 266(5192): 1821-1828.
Helleday, T., Petermann, E., Lundin, C., Hodgson, B., and Sharma, R. A. 2008. DNA repair pathways as targets for cancer therapy. Nat Rev Cancer 8(3): 193-204.
Hilmi, C., Larribere, L., Giuliano, S., Bille, K., Ortonne, J. P., Ballotti, R., and Bertolotto, C. 2008. IGF1 promotes resistance to apoptosis in melanoma cells through an increased expression of BCL2, BCL-X(L), and survivin. J Invest Dermatol 128(6): 1499-1505.
Huang, T. T., Wuerzberger-Davis, S. M., Seufzer, B. J., Shumway, S. D., Kurama, T., Boothman, D. A., and Miyamoto, S. 2000. NF-kappaB activation by camptothecin. A linkage between nuclear DNA damage and cytoplasmic signaling events. J Biol Chem 275(13): 9501-9509.
Imbert, V., Rupec, R. A., Livolsi, A., Pahl, H. L., Traenckner, E. B., Mueller-Dieckmann, C., Farahifar, D., Rossi, B., Auberger, P., Baeuerle, P. A., and Peyron, J. F. 1996. Tyrosine phosphorylation of I kappa B-alpha activates NF-kappa B without proteolytic degradation of I kappa B-alpha. Cell 86(5): 787-798.
Jane-Valbuena, J., Widlund, H. R., Perner, S., Johnson, L. A., Dibner, A. C., Lin, W. M., Baker, A. C., Nazarian, R. M., Vijayendran, K. G., Sellers, W. R., Hahn, W. C., Duncan, L. M., Rubin, M. A., Fisher, D. E., and Garraway, L. A. 2010. An oncogenic role for ETV1 in melanoma. Cancer Res 70(5): 2075-2084.
Khaled, M., Larribere, L., Bille, K., Ortonne, J. P., Ballotti, R., and Bertolotto, C. 2003. Microphthalmia associated transcription factor is a target of the phosphatidylinositol-3-kinase pathway. J Invest Dermatol 121(4): 831-836.
Kolthur-Seetharam, U., Dantzer, F., McBurney, M. W., de Murcia, G., and Sassone-Corsi, P. 2006. Control of AIF-mediated cell death by the functional interplay of SIRT1 and PARP-1 in response to DNA damage. Cell Cycle 5(8): 873-877.
Krtolica, A., Parrinello, S., Lockett, S., Desprez, P. Y., and Campisi, J. 2001. Senescent fibroblasts promote epithelial cell growth and tumorigenesis: a link between cancer and aging. Proc Natl Acad Sci USA 98(21): 12072-12077.
Kuilman, T., Michaloglou, C., Vredeveld, L. C., Douma, S., van Doom, R., Desmet, C. J., Aarden, L. A., Mooi, W. J., and Peeper, D. S. 2008. Oncogene-induced senescence relayed by an interleukin-dependent inflammatory network. Cell 133(6): 1019-1031.
Kuilman, T. and Peeper, D. S. 2009. Senescence-messaging secretome: SMS-ing cellular stress. Nat Rev Cancer 9(2): 81-94.
Larribere, L., Hilmi, C., Khaled, M., Gaggioli, C., Bille, K., Auberger, P., Ortonne, J. P., Ballotti, R., and Bertolotto, C. 2005. The cleavage of microphthalmia-associated transcription factor, MITF, by caspases plays an essential role in melanocyte and melanoma cell apoptosis. Genes Dev 19(17): 1980-1985.
Lawrence, T. 2009. The nuclear factor NF-kappaB pathway in inflammation. Cold Spring Harb Perspect Biol 1(6): a001651.
Lev, D. C., Ruiz, M., Mills, L., McGary, E. C., Price, J. E., and Bar-Eli, M. 2003. Dacarbazine causes transcriptional up-regulation of interleukin 8 and vascular endothelial growth factor in melanoma cells: a possible escape mechanism from chemotherapy. Mol Cancer Ther 2(8): 753-763.
Li, N., Banin, S., Ouyang, H., Li, G. C., Courtois, G., Shiloh, Y., Karin, M., and Rotman, G. 2001. ATM is required for IkappaB kinase (IKKk) activation in response to DNA double strand breaks. J Biol Chem 276(12): 8898-8903.
Li, N. and Karin, M. 1998. Ionizing radiation and short wavelength UV activate NF-kappaB through two distinct mechanisms. Proc Natl Acad Sci USA 95(22): 13012-13017.
Liu, D. and Hornsby, P. J. 2007. Senescent human fibroblasts increase the early growth of xenograft tumors via matrix metalloproteinase secretion. Cancer Res 67(7): 3117-3126.
Liu, F., Fu, Y., and Meyskens, F. L., Jr. 2009. MiTF regulates cellular response to reactive oxygen species through transcriptional regulation of APE-1/Ref-1. J Invest Dermatol 129(2): 422-431.
Mhaidat, N. M., Zhang, X. D., Allen, J., Avery-Kiejda, K. A., Scott, R. J., and Hersey, P. 2007. Temozolomide induces senescence but not apoptosis in human melanoma cells. Br J Cancer 97(9): 1225-1233.
Piret, B., Schoonbroodt, S., and Piette, J. 1999. The ATM protein is required for sustained activation of NF-kappaB following DNA damage. Oncogene 18(13): 2261-2271.
Schmitt, C. A., Fridman, J. S., Yang, M., Lee, S., Baranov, E., Hoffman, R. M., and Lowe, S. W. 2002. A senescence program controlled by p53 and p16INK4a contributes to the outcome of cancer therapy. Cell 109(3): 335-346.
Schneider, G., Henrich, A., Greiner, G., Wolf, V., Lovas, A., Wieczorek, M., Wagner, T., Reichardt, S., von Werder, A., Schmid, R. M., Weih, F., Heinzel, T., Saur, D., and Kramer, O. H. 2010. Cross talk between stimulated NF-kappaB and the tumor suppressor p53. Oncogene 29(19): 2795-2806.
Stathopoulos, G. T., Psallidas, I., Moustaki, A., Moschos, C., Kollintza, A., Karabela, S., Porfyridis, I., Vassiliou, S., Karatza, M., Zhou, Z., Joo, M., Blackwell, T. S., Roussos, C., Graf, D., and Kalomenidis, I. 2008. A central role for tumor-derived monocyte chemoattractant protein-1 in malignant pleural effusion. J Natl Cancer Inst 100(20): 1464-1476.
Stilmann, M., Hinz, M., Arslan, S. C., Zimmer, A., Schreiber, V., and Scheidereit, C. 2009. A nuclear poly(ADP-ribose)-dependent signalosome confers DNA damage-induced IkappaB kinase activation. Mol Cell 36(3): 365-378.
Strub, T., Giuliano, S., Ye, T., Bonet, C., Keime, C., Kobi, D., Legras, S., Cormont, M., Ballotti, R., Bertolotto, C., and Davidson, I. 2010. Essential role of Microphthalmia transcription factor for DNA replication, mitosis and genomic stability in melanoma. In press.
te Poele, R. H., Okorokov, A. L., Jardine, L., Cummings, J., and Joel, S. P. 2002. DNA damage is able to induce senescence in tumor cells in vitro and in vivo. Cancer Res 62(6): 1876-1883.
Tentori, L., Muzi, A., Dorio, A. S., Scarsella, M., Leonetti, C., Shah, G. M., Xu, W., Camaioni, E., Gold, B., Pellicciari, R., Dantzer, F., Zhang, J., and Graziani, G. 2010. Pharmacological inhibition of poly(ADP-ribose) polymerase (PARP) activity in PARP-1 silenced tumour cells increases chemosensitivity to temozolomide and to a N3-adenine selective methylating agent. Curr Cancer Drug Targets 10(4): 368-383.
Valenzuela, M. T., Guerrero, R., Nunez, M. I., Ruiz De Almodovar, J. M., Sarker, M., de Murcia, G., and Oliver, F. J. 2002. PARP-1 modifies the effectiveness of p53-mediated DNA damage response. Oncogene 21(7): 1108-1116.
Veuger, S. J., Hunter, J. E., and Durkacz, B. W. 2009. Ionizing radiation-induced NF-kappaB activation requires PARP-1 function to confer radioresistance. Oncogene 28(6): 832-842.
Wajapeyee, N., Serra, R. W., Zhu, X., Mahalingam, M., and Green, M. R. 2008. Oncogenic BRAF induces senescence and apoptosis through pathways mediated by the secreted protein IGFBP7. Cell 132(3): 363-374.
Wellbrock, C., Rana, S., Paterson, H., Pickersgill, H., Brummelkamp, T., and Marais, R. 2008. Oncogenic BRAF regulates melanoma proliferation through the lineage specific factor MITF. PLoS One 3(7): e2734.
Wesierska-Gadek, J., Ranftler, C., and Schmid, G. 2005. Physiological ageing: role of p53 and PARP-1 tumor suppressors in the regulation of terminal senescence. J Physiol Pharmacol 56 Suppl 2: 77-88.
Wieler, S., Gagne, J. P., Vaziri, H., Poirier, G. G., and Benchimol, S. 2003. Poly(ADP-ribose) polymerase-1 is a positive regulator of the p53-mediated G1 arrest response following ionizing radiation. J Biol Chem 278(21): 18914-18921.
Wu, Z. H., Shi, Y., Tibbetts, R. S., and Miyamoto, S. 2006. Molecular linkage between the kinase ATM and NF-kappaB signaling in response to genotoxic stimuli. Science 311(5764): 1141-1146.
Yang, J., Amiri, K. I., Burke, J. R., Schmid, J. A., and Richmond, A. 2006. BMS-345541 targets inhibitor of kappaB kinase and induces apoptosis in melanoma: involvement of nuclear factor kappaB and mitochondria pathways. Clin Cancer Res 12(3 Pt 1): 950-960.
Yang, J., Pan, W. H., Clawson, G. A., and Richmond, A. 2007. Systemic targeting inhibitor of kappaB kinase inhibits melanoma tumor growth. Cancer Res 67(7): 3127-3134.
Yang, J., Splittgerber, R., Yull, F. E., Kantrow, S., Ayers, G. D., Karin, M., and Richmond, A. 2010. Conditional ablation of Ikkb inhibits melanoma tumor development in mice. J Clin Invest 120(7): 2563-2574.
Zaidi, M. R., Davis, S., Noonan, F. P., Graff-Cherry, C., Hawley, T. S., Walker, R. L., Feigenbaum, L., Fuchs, E., Lyakh, L., Young, H. A., Hornyak, T. J., Arnheiter, H., Trinchieri, G., Meltzer, P. S., De Fabo, E. C., and Merlino, G. 2011. Interferon-gamma links ultraviolet radiation to melanomagenesis in mice. Nature.
Zhu, X., Fujita, M., Snyder, L. A., and Okada, H. 2010. Systemic delivery of neutralizing antibody targeting CCL2 for glioma therapy. J Neurooncol.

1. A method for predicting the tumor size evolution and/or the onset of metastasis in a patient suffering from a cancer, comprising the step of measuring the level of expression of at least one gene selected from the group consisting of CCL2, IL6, Cyr61, IGFBP7, CCL8, PAI1, OPG, MMP2 and UPAR genes in cancer cells of said patient.

2. A method according to claim 1, wherein said cancer is a melanoma.

3. A product comprising (i) a chemotherapeutic agent and (ii) at least an active compound chosen from CCL2 inhibitors, CCR2 inhibitors, NF-κB inhibitors, PARP-1 inhibitors and ATM inhibitors, as a combined preparation for simultaneous, separate or sequential use for inhibiting tumor development caused by tumor cell senescence induced by said chemotherapeutic agent.

4. A method for inhibiting tumor development tumor growth, or tumor metastasis in a subject, comprising administering to said subject a pharmaceutical composition comprising (i) said chemotherapeutic agent and (ii) at least an active compound chosen from CCL2 inhibitors, CCR2 inhibitors, NF-κB inhibitors, PARP-1 inhibitors and ATM inhibitors.

5. A product according to claim 3, wherein said active compound (ii) is chosen from CCL2 antagonists, NF-κB antagonists, PARP-1 antagonists and ATM antagonists.

6. A product according to claim 5, wherein said active compound (ii) is chosen from antibodies, sulfasalazine, BMS-345541, bortezomid, 3-aminobenzamide, Iniparib, Olaparib, ABT-888, AG014699, CEP 9722, MK 4827, KU-0059436, LT-673, KU55933 and caffeine.

7. A product according to claim 6, wherein said active compound (ii) is chosen from CCL2 antagonists.

8. A product according to claim 3, wherein said active compound (ii) is chosen from CCL2 expression inhibitors, NF-κB expression inhibitors, PARP-1 expression inhibitors and ATM expression inhibitors.

9. A product according to claim 8, wherein said active compound (ii) is chosen from antisense oligonucleotides, siRNAs, shRNAs, ribozymes and DNAzymes.

10. A product according to claim 9, wherein said active compound (ii) is chosen from CCL2 siRNAs.

11. A product according to claim 3 wherein said chemotherapeutic agent is selected from temozolomide, fotemustine, dacarbazine, 5-fluorouracil, bevacizumab, irinotecan, SN38, oxaliplatin, cetuximab, panitumumab, leucovorine, bortezomib and capecitabine.

12. The method of claim 4 wherein said tumor development is caused by tumor cell senescence induced by said chemotherapeutic agent.

13. A method for monitoring the response to a chemotherapeutic agent of a patient suffering from a cancer, comprising the step of measuring the level of expression of at least one gene selected from the group consisting of CCL2, Cyr61, IL6, IGFBP7, CCL8, PAI1, OPG, MMP2 and UPAR genes in cancer cells of said patient.

14. A method according to claim 13, wherein said cancer is a melanoma.

15. A method according to claim 4, wherein said active compound (ii) is chosen from CCL2 antagonists, NF-κB antagonists, PARP-1 antagonists and ATM antagonists.

16. A method according to claim 15, wherein said active compound (ii) is chosen from antibodies, sulfasalazine, BMS-345541, bortezomid, 3-aminobenzamide, Iniparib, Olaparib, ABT-888, AG014699, CEP 9722, MK 4827, KU-0059436, LT-673, KU55933 and caffeine.

17. A method according to claim 16, wherein said active compound (ii) is chosen from CCL2 antagonists.

18. A method according to claim 4, wherein said active compound (ii) is chosen from CCL2 expression inhibitors, NF-κB expression inhibitors, PARP-1 expression inhibitors and ATM expression inhibitors.

19. A method according to claim 8, wherein said active compound (ii) is chosen from antisense oligonucleotides, siRNAs, shRNAs, ribozymes and DNAzymes.

20. A method according to claim 19, wherein said active compound (ii) is chosen from CCL2 siRNAs.

21. A method according to claim 4, wherein said chemotherapeutic agent is selected from temozolomide, fotemustine, dacarbazine, 5-fluorouracil, bevacizumab, irinotecan, SN38, oxaliplatin, cetuximab, panitumumab, leucovorine, bortezomib and capecitabine.

22. A method according to claim 15, wherein said chemotherapeutic agent is selected from temozolomide, fotemustine, dacarbazine, 5-fluorouracil, bevacizumab, irinotecan, SN38, oxaliplatin, cetuximab, panitumumab, leucovorine, bortezomib and capecitabine.

23. A method according to claim 16, wherein said chemotherapeutic agent is selected from temozolomide, fotemustine, dacarbazine, 5-fluorouracil, bevacizumab, irinotecan, SN38, oxaliplatin, cetuximab, panitumumab, leucovorine, bortezomib and capecitabine

24. A method according to claim 17, wherein said chemotherapeutic agent is selected from temozolomide, fotemustine, dacarbazine, 5-fluorouracil, bevacizumab, irinotecan, SN38, oxaliplatin, cetuximab, panitumumab, leucovorine, bortezomib and capecitabine

25. A method according to claim 18, wherein said chemotherapeutic agent is selected from temozolomide, fotemustine, dacarbazine, 5-fluorouracil, bevacizumab, irinotecan, SN38, oxaliplatin, cetuximab, panitumumab, leucovorine, bortezomib and capecitabine

26. A method according to claim 19, wherein said chemotherapeutic agent is selected from temozolomide, fotemustine, dacarbazine, 5-fluorouracil, bevacizumab, irinotecan, SN38, oxaliplatin, cetuximab, panitumumab, leucovorine, bortezomib and capecitabine

27. A method according to claim 20, wherein said chemotherapeutic agent is selected from temozolomide, fotemustine, dacarbazine, 5-fluorouracil, bevacizumab, irinotecan, SN38, oxaliplatin, cetuximab, panitumumab, leucovorine, bortezomib and capecitabine
